(19) United States
(12) Patent Application Publication
(10) Pub. No.: US 2011/0144431 A1
(43) Pub. Date: Jun. 16, 2011

(54) SYSTEM AND METHOD FOR CONTROLLING USE OF CAPSULE ENDOSCOPES

(76) Inventors: Rainer Graumann, Hoechstadt (DE); Rene Hempel, Magdeburg (DE); Rainer Kuth, Hoechstadt (DE); Seiichi Tadano, Tokyo (JP)

(21) Appl. No.: 12/637,949

(22) Filed: Dec. 15, 2009

(51) Int. Cl. A61B 1/04; A61B 1/00

(52) U.S. Cl. 600/109; 600/118

(57) ABSTRACT

In a method or system for limiting use of capsule endoscopes by a capsule endoscope maagnetic steering system used by a customer, the capsule endoscope is provided with a use code stored with the capsule. The use code is read and analyzed to determine whether or not the customer's capsule endoscope magnetic steering system will be enabled or disabled for use with the capsule endoscope from which the use code was read.

### BACKGROUND

The disclosed embodiment concerns the use of capsule endoscopes which are typically ingested by a patient and which traverse the gastrointestinal tract of the patient, and which are then voided in a natural manner.

Capsule endoscopes are well known for testing for therapy of a gastrointestinal tract where, for example, an image sensor unit may capture views of the interior of the body. The patient swallows the capsules and they traverse the gastrointestinal tract. After eight hours, for example, the endoscopy capsule is voided naturally via the anus.

Capsule endoscopes known today are relatively expensive and normally may be used only once.

The capsule endoscope traverses the gastrointestinal tract of a patient and obtains images by means of an image sensor, for example, which are transmitted by a wireless transmission to a receiver.

It is known to provide magnets within the capsule endoscopes within a shell or casing of the capsule. By use of a large magnet creating a magnetic field, the capsule is then steered within the patient. The capsule endoscope magnetic steering system is a relatively expensive system, since the magnet required is relatively large. Some customers for the capsules such as physicians, physician groups, hospitals, etc. may have a choice to invest in the relatively expensive capsule endoscope magnetic steering system and also purchase the capsules for use in that system. Other customers may decide not to make an investment in the capsule endoscope magnetic steering system but prefer to either lease the system, or have free use of the system provided that they pay a higher price for the capsule endoscopes.

Also, it may be desirable to provide free use of the magnetic steering system together with the relatively lower priced capsule endoscopes to certain customers on a promotional and/or charitable basis such as medical schools or the like. Also in certain regions of the world it may be desirable to provide the steering system and the less expensive capsules for humanitarian and/or charitable reasons.

For the first alternative listed above where the customer buys or leases the system, he may receive the capsules at a discounted price in view of his investment or lease payments for the relatively expensive capsule endoscope magnetic steering system. There is, of course, the possibility and risk that those customers might sell the capsules which they obtained at a lower price to the other customers who did not invest or lease the capsule endoscope magnetic steering system, but who would be required normally to pay a higher price for the capsules given free use of the capsule endoscope magnetic steering system.

### SUMMARY

In a method or system for limiting use of capsule endoscopes by a capsule endoscope magnetic steering system used by a customer, the capsule endoscope is provided with a use code stored with the capsule. The use code is read and analyzed to determine whether or not the customer's capsule endoscope magnetic steering system will be enabled or disabled for use with the capsule endoscope from which the use code was read.

### BRIEF DESCRIPTION OF THE DRAWINGS

FIG. 1 is a perspective view of a steerable capsule endoscope according to the prior art;

FIG. 2 is a perspective view of the capsule endoscope magnetic steering system according to the prior art for steering the capsule of FIG. 1 but with a use code transmitter/receiver mounted thereon together with associated enable/disable logic according to the present first preferred embodiment;

FIG. 3A is a schematic block diagram view of a capsule endoscope magnetic steering system and method according to a first preferred embodiment which may limit use of a capsule endoscope depending upon a use code;

FIG. 3B is a schematic block diagram of a second embodiment of the method and system for limiting use of the capsule endoscope;

FIG. 3C is a schematic block diagram of a third embodiment for limiting use of the capsule endoscope; and

FIG. 4 is a flowchart showing method steps according to the preferred embodiments for limiting use of the capsule endoscope.

### DESCRIPTION OF THE PREFERRED EMBODIMENTS

For the purposes of promoting an understanding of the principles of the invention, reference will now be made to the preferred embodiments/best mode illustrated in the drawings and specific language will be used to describe the same. It will nevertheless be understood that no limitation of the scope of the invention is thereby intended, and such alterations and further modifications in the illustrated device and such further applications of the principles of the invention as illustrated as would normally occur to one skilled in the art to which the invention relates are included.

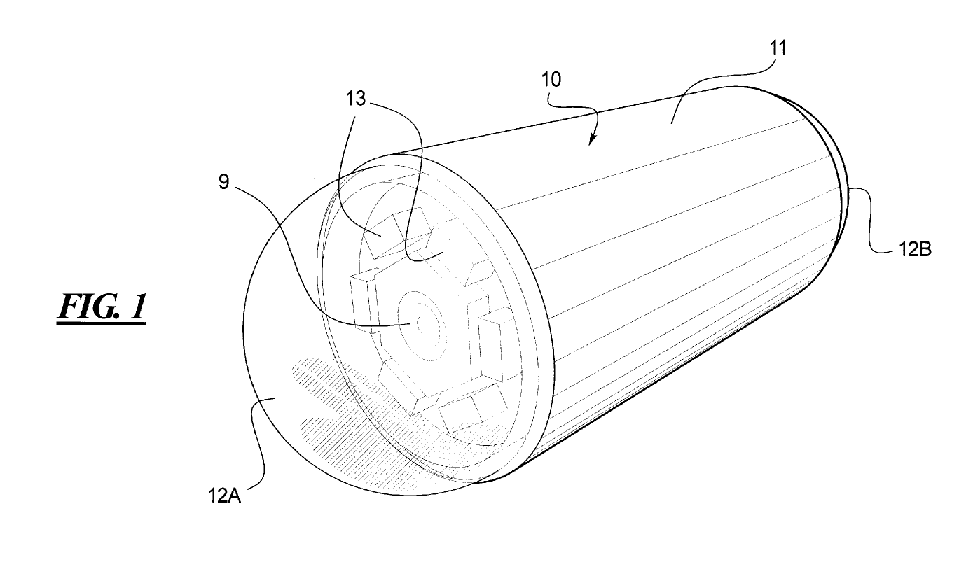

FIG. 1 shows a perspective view of the prior art capsule endoscope 10 having an outer sheath or casing 11 and hemispherical clear plastic ends 12A and 12B. An image sensor 9 is provided internally surrounded by a plurality of magnets 13 used for steering the capsule and disposed at a periphery near the outer casing 11.

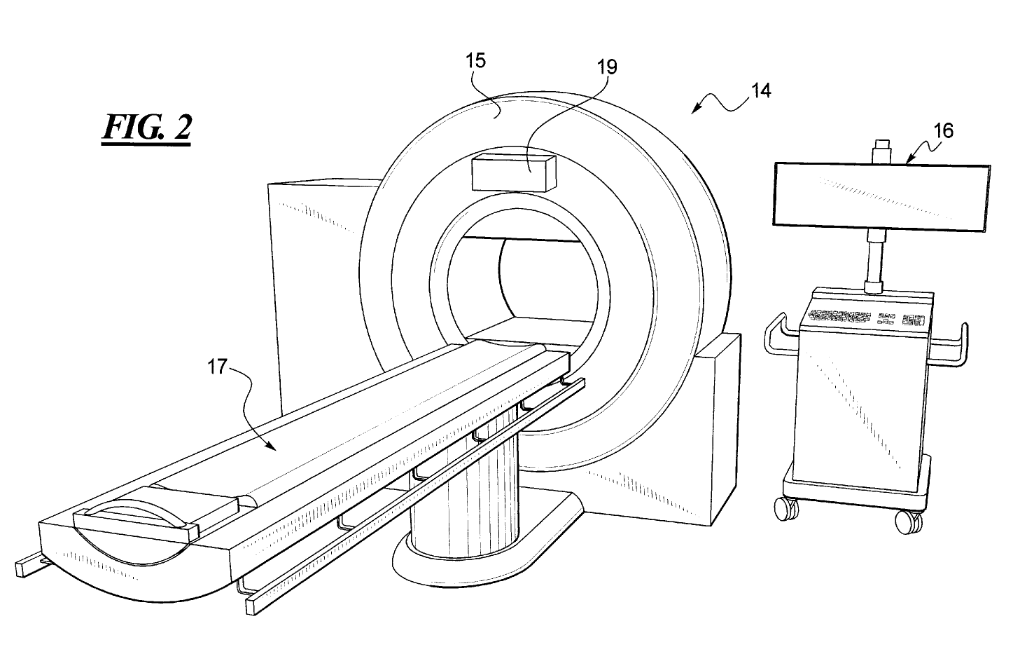

FIG. 2 is a perspective view of the prior art capsule endoscope magnetic steering system 14 having a large cylindrical steering magnet 15, steering and video monitor control unit 16, and bed 17 for the patient. The capsule endoscope magnetic steering system steers the capsule endoscope through the patient and also provides the image monitor at control unit 16. To this prior art unit a use code transmitter/receiver 19 with use code logic has been added according to the preferred embodiments described hereafter.

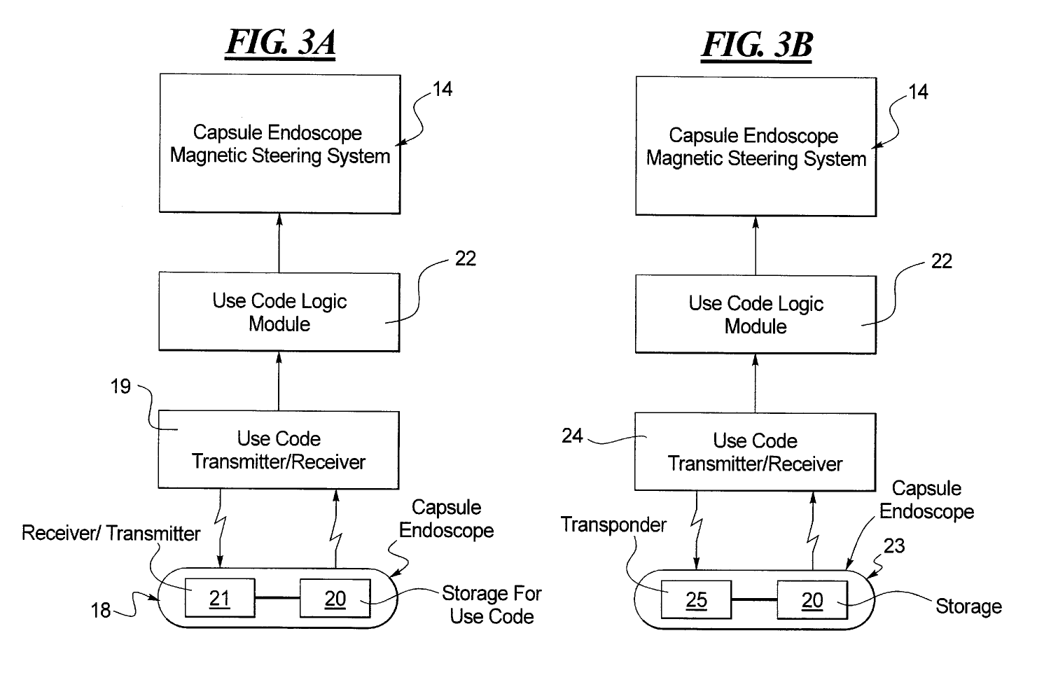

FIG. 3A shows the capsule endoscope 18 having an RF receiver/transmitter 21 therein powered by an internal capsule battery and receiving use code inquiry signals from use code transmitter/receiver 19 and for transmitting signals to use code transmitter/receiver 19 in response to the inquiry signals. The receiver/transmitter 21 retrieves a use code stored in the storage 20 and transmits the use code to the use code transmitter/receiver 19. The inquiry signals for the use code are sent from the use code transmitter/receiver 19 shown in FIG. 2 when the patient having the ingested capsule endoscope as lying on the patient bed 17. Alternatively, the inquiry signals could be sent to the capsule endoscope prior to introduction into the patient.

The use code transmitter/receiver 19 forwards the use code to a use code logic module 22 which then sends an enable or disable signal to the capsule endoscope magnetic steering system 14 with its control unit 16. As shown in FIG. 2 use code transmitter/receiver 19 is preferably mounted near or on the magnet 15 to monitor the capsule ingested by the patient but prior to entry of the patient into the magnetic field provided by the steering magnet 15.

The transmitter in FIG. 3A is powered by a battery already provided in the capsule endoscope for transmitting images.

Alternatively, in a second embodiment shown in FIG. 3B, the storage 20 may be connected to a passive transponder 25 in capsule endoscope 23 receiving inquiry RF signals from the transmitter/receiver 24 and then using those signals to power the transponder 25 which then sends RF response signals with the stored use code, which is then passed on to the use code logic module 22 for triggering the enable or disable signal to the capsule endoscope magnetic steering system 14 with control unit 16. Alternatively, the inquiry signals could be sent to the capsule prior to the introduction into the patient.

In a third embodiment shown in FIG. 3C, the use code may be stored with scanner readable identification 27 such as a barcode, for example, on an outside surface of the casing 26A of capsule endoscope 27. Alternatively if the casing is transparent, the identification could be stored inside of the capsule and read through the clear outer casing. The use code scanner/receiver 28 transmits scanning light which is reflected and picked up by the use code scanner/receiver 28. The use code scanner/receiver 28 then sends the detected use code to the use code logic module 22 which then sends the enable or disable signal to the capsule endoscope magnetic steering system 14 with control unit 16. In this embodiment, the capsule would be scanned prior to introduction into the patient.

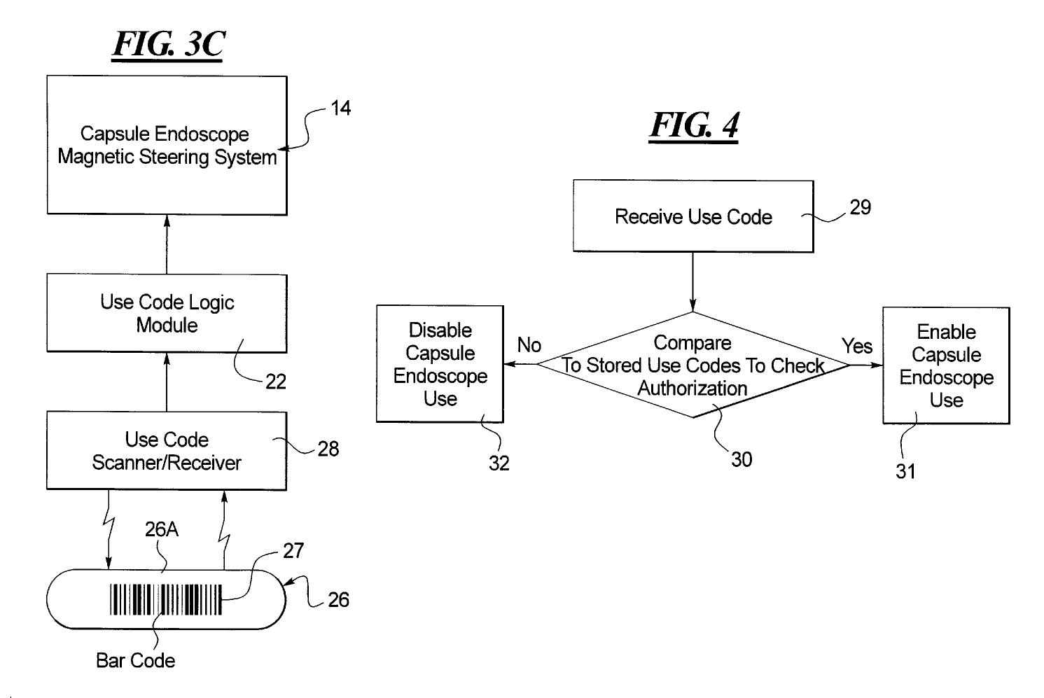

FIG. 4 shows the method steps in the use code logic module 22. The use code is received at step 29. At step 30, the use code is compared to stored use codes to determine whether or not the use code is an authorized use code to enable operation of the capsule endoscope magnetic steering system 14. If yes, it is enabled at 31. If no, the capsule endoscope magnetic steering system is disabled at 32.

Typically, use codes which will be authorized to enable the capsule endoscope use by the capsule endoscope magnetic steering system 14 will be for those capsules which were sold at a relatively low price to customers who had purchased or leased the capsule endoscope magnetic steering system or for promotional and/or charitable (humanitarian) customers such as medical schools for the like.

On the other hand, for those customers who have not purchased the capsule endoscope magnetic steering system who have been given free use of a “loaner system”, but who purchase more expensive capsules, the use code logic with such a loaner steering system would preclude use of relatively low price capsules by disabling the capsule endoscope magnetic steering system for use with such lower price capsules.

Customers deemed to be authorized to use the relatively lower price capsules could be also in certain regions based on charitable and/or humanitarian needs for those regions.

The use code logic module may also authorize certain use codes for use in a given time period so that a customer may use a certain number of the relatively lower priced capsules for a certain time period. Also use may be permitted for a fixed number of lower price capsules, then thereafter only the use of relatively higher price capsules may be permitted based on the use code logic and use codes provided.

The use codes may be combinations of numbers and/or letters, and/or may be a serial number of each individual capsule.

While preferred embodiments have been illustrated and described in detail in the drawings and foregoing description, the same are to be considered as illustrative and not restrictive in character, it being understood that only the preferred embodiments have been shown and described and that all changes and modifications that come within the spirit of the invention both now or in the future are desired to be protected.

I claim as my invention:

1. A method for limiting use of capsule endoscopes by a capsule endoscope magnetic steering system used by a customer, comprising the steps of:
providing the capsule endoscope with a use code with the capsule; and
at a location of the customer where the capsule endoscope is to be magnetically steered through a patient with the capsule endoscope magnetic steering system, reading the use code with the use code reader and analyzing the use code to determine whether or not the customer's capsule endoscope magnetic steering system will be enabled or disabled for use with the capsule endoscope from which the use code was read.

2. A method of claim 1 wherein the use code is stored in a storage in the capsule endoscope and transmitted with a transmitter powered by a battery in the capsule to a use code receiver and then to a use code logic module.

3. A method of claim 1 wherein the use code is stored in a storage in the capsule endoscope and the capsule endoscope has a passive transponder which reacts to a received signal and then transmits to a use code receiver and then to a use code logic module.

4. A method of claim 1 wherein the use code is stored by an identification at the capsule which are scanned by a use code scanner to determine the use by a use code receiver and then to a use code logic module.

5. A method of claim 4 wherein the identification comprises a barcode.

6. A method of claim 4 wherein the identification is on an outside of the casing of a capsule.

7. A method of claim 4 wherein the identification is at an inside of the casing of the capsule.

8. A method of claim 1 wherein a use code logic analyzes the use code and enables the capsule endoscope magnetic steering system for use with the capsule if it is a relatively low price capsule since the customer is at least one of a purchaser, lesser, promotional, and charitable user of the capsule endoscope magnetic steering system, but otherwise disables use of the capsule endoscope steering system.

9. A method of claim 1 wherein a use code logic analyzes the use code and enables the capsule endoscope magnetic steering system for use with the capsule if a certain time period is not yet passed, and after said time period operation of the steering system is disabled.

10. A method of claim 1 wherein a use code logic analyzes the use code and enables the capsule endoscope magnetic steering system for use with the capsule if a certain number of said capsules used by said customer has not yet been reached, and after said certain number has been reached, disabling the magnetic steering system.

11. A system for limiting use of capsule endoscopes, comprising:
a capsule endoscope having at least one magnet therein and an image sensor, and a transmitter to transmit images;
a capsule endoscope magnetic steering system for steering the capsule through a patient of a customer who is supplying the capsule to the patient;
said capsule endoscope having a use code stored with the capsule and transmission system for sending the use code;
at a location of said customer where said capsule endoscope magnetic steering system is provided a receiver for receiving the use code; and
a logic module connected to receive the use code to analyze the use code to determine whether or not the customer's capsule endoscope magnetic steering system will be enabled or disabled for use with the capsule endoscope from which the use code was read.

12. A system of claim 11 wherein the capsule has a transmitter powered by a battery in the capsule.

13. A system of claim 11 wherein the use code is transmitted with a passive transponder.

14. A system of claim 11 wherein the use code is stored as an identification in the capsule which are scanned by a use code scanner.

15. A system of claim 11 wherein the identification is on an a casing of the capsule.

16. A system of claim 11 wherein the identification is at an inside of said capsule.

17. A system of claim 11 wherein the use code logic module analyzes the use code and enables the capsule endoscope magnetic steering system for use with the capsule if it is a relatively low price capsule where the customer is at least one of a purchaser, lesser, promotional, and charitable user of the steering system, but otherwise disables use of the capsule endoscope magnetic steering system.

18. A system of claim 11 wherein a use code logic analyzes the use code and enables the capsule endoscope magnetic steering system for use with the capsule if a certain time period is not yet passed, and after said time period operation of the steering system is disabled.

19. A method for limiting use of capsule endoscopes by a capsule endoscope magnetic steering system used by a customer, comprising the steps of:
providing the capsule endoscope with a use code with the capsule; and
reading the use code with the use code reader and analyzing the use code to determine whether or not the customer's capsule endoscope magnetic steering system will be enabled or disabled for use with the capsule endoscope from which the use code was read.
